(19) United States
(12) Patent Application Publication
(10) Pub. No.: US 2019/0304569 A1
(43) Pub. Date: Oct. 3, 2019

(54) DRUG TARGETING OF HUMAN INDOLEAMINE 2,3-DIOXYGENASE

(71) Applicant: Albert Einstein College of Medicine, Bronx, NY (US)

(72) Inventors: Syun-Ru Yeh, Fort Lee, NJ (US); Ariel Lewis-Ballester, Bronx, NY (US); Khoa Ngoc Pham, Miami, FL (US)

(21) Appl. No.: 16/432,831

(22) Filed: Jun. 5, 2019

Related U.S. Application Data
(63) Continuation of application No. PCT/US2017/064460, filed on Dec. 4, 2017.
(60) Provisional application No. 62/430,408, filed on Dec. 6, 2016.

(51) Int. Cl. G16B 15/30; C12N 9/02; G16B 40/00; A61K 31/352

(52) U.S. Cl. CPC G16B 15/30; C12N 9/0069; C12Y 113/11011; A61K 31/352; G16B 40/00

(57) ABSTRACT

A system and a method for identifying novel hIDO inhibitors.

### CROSS-REFERENCE TO RELATED APPLICATIONS

This application claims benefit of U.S. Provisional Application No. 62/430,408, filed Dec. 6, 2016, the contents of which are hereby incorporated by reference.

### STATEMENT OF GOVERNMENT SUPPORT

This invention was made with government support under grant number GM086482 awarded by the National Institutes of Health. The government has certain rights in the invention.

### MEGATABLES INCOPORATION

The “.txt” MEGATABLES 1, 2 and 3 filed by EFS in connection with the priority application, U.S. Provisional Application No. 62/430,408, filed Dec. 6, 2016, and which are, respectively (i) IDEWTCNTRP_ref13_May24.txt, 978 kilobytes in size, created on Nov. 23, 2016; (ii) IDES167H17_refine_37-may23.txt, 1 MB in size, created on Nov. 23, 2016, and (iii), F270G30day10_refine_11.txt, 986 kilobytes in size created on Nov. 23, 2016, are each hereby incorporated by reference.

### BACKGROUND OF THE INVENTION

Throughout this application various publications are referred to. Full citations for the references may be found at the end of the specification. The disclosures of these publications are hereby incorporated by reference in their entirety into the subject application to more fully describe the art to which the subject invention pertains.

Human Indoleamine 2,3-dioxygenase (hIDO) is an immunosuppressive enzyme, which is induced in a wide range of cancers (1). It is critical for cancer immune escape, as well as transplant and maternal-fetal tolerance (2). Consequently, hIDO has been identified as an important therapeutic target (3). Recent advances in the development of hIDO-inhibitors as immunotherapy for cancer have trigger several million-to-billion dollar deals in pharmaceutical industry to license hIDO-inhibitors.

Owing to poor structural information, so far all the structural-based drug design of hIDO inhibitors is limited to substrate analogs that target the active site (4). The use of the active site as the drug target poses a variety of problems. First, the active site exhibits unusual structural flexibility, hindering the utility of structure-based drug design. Second, drugs bound in the active site can potentially coordinate to or interact with the heme iron, leading to poor selectivity against other heme-containing proteins, such as P450's. In addition, the potential interaction of drugs with the heme iron causes strong sensitivity of hIDO to the electronic nature of drug candidates, making in-silico drug design difficult. Last, but not the least, the active site does not only bind Trp, but it also binds O₂ (on the heme iron), making the determination of the inhibition modes cumbersome

The present invention addresses the need for a new method and system for identifying novel hIDO inhibitors.

### SUMMARY OF THE INVENTION

Provided is a non-transitory computer-readable medium coupled to the one or more data processing apparatus having instructions stored thereon which, when executed by the one or more data processing apparatus, cause the one or more data processing apparatus to perform a method comprising:
providing a crystal coordinate structure of the small molecule binding site on a human indoleamine 2,3-dioxygenase (hIDO) enzyme, wherein the small molecule binding site is not a hIDO active site;
contacting the small molecule binding site in silico with a candidate small organic molecule structure; and
determining if the candidate small organic molecule structure binds the small molecule binding site at a free energy level below a predetermined free energy level,
wherein a candidate small organic molecule structure that binds the small molecule binding site at a free energy level below the predetermined free energy level is identified and detected as a molecule which binds to a small molecule binding site on a human indoleamine 2,3-dioxygenase (hIDO) enzyme.

Also provided is a system for identifying a cognate ligand molecule for a known biological receptor comprised of amino acid residues, comprising:
one or more data processing apparatus;
a graphical user interface; and
a non-transitory computer-readable medium coupled to the one or more data processing apparatus having instructions stored thereon which, when executed by the one or more data processing apparatus, cause the one or more data processing apparatus to perform a method comprising:
providing a crystal coordinate structure of the small molecule binding site on a human indoleamine 2,3-dioxygenase (hIDO) enzyme, wherein the small molecule binding site is not a hIDO active site;
contacting the small molecule binding site in silico with a candidate small organic molecule structure; and
determining if the candidate small organic molecule structure binds the small molecule binding site at a free energy level below a predetermined free energy level,
wherein a candidate small organic molecule structure that binds the small molecule binding site at a free energy level below the predetermined free energy level is identified and detected as a molecule which binds to a small molecule binding site on a human indoleamine 2,3-dioxygenase (hIDO) enzyme.

Also provided is a method of detecting a molecule which binds to a small molecule binding site on a human indoleamine 2,3-dioxygenase (hIDO) enzyme, wherein the small molecule binding site is not a hIDO active site, comprising:
providing a crystal coordinate structure of the small molecule binding site on a human indoleamine 2,3-dioxygenase (hIDO) enzyme, wherein the small molecule binding site is not a hIDO active site;
contacting the small molecule binding site in silico with a candidate small organic molecule structure; and
determining if the candidate small organic molecule structure binds the small molecule binding site at a free energy level below a predetermined free energy level,
wherein a candidate small organic molecule structure that binds the small molecule binding site at a free energy level below the predetermined free energy level is identified and detected as a molecule which binds to a small molecule binding site on a human indoleamine 2,3-dioxygenase (hIDO) enzyme.

A method of activating or increasing activation of human indoleamine 2,3-dioxygenase (hIDO) enzyme comprising contacting the hIDO with an amount of Apigenin, Baicalein or Chrysin having the structures as set forth in Table 1, or a pharmaceutical salt of one thereof, in an amount effective to activating or increasing activation of hIDO.

A method of effecting immunosuppression in a subject comprising administering an amount of Apigenin, Baicalein or Chrysin having the structures as set forth in Table 1, or a pharmaceutical salt of one thereof, in an amount effective to activate or increasing activation of hIDO.

A method of treating an autoimmune disease or inhibiting an organ transplant rejection in a subject comprising administering an amount of Apigenin, Baicalein or Chrysin having the structures as set forth in Table 1, or a pharmaceutical salt of one thereof, in an amount effective to treat an autoimmune disease or inhibit an organ transplant rejection.

A pharmaceutical composition for activating or increasing activation of human indoleamine 2,3-dioxygenase comprising an amount of Apigenin, Baicalein or Chrysin having the structures as set forth in Table 1, or a pharmaceutical salt of one thereof, and a pharmaceutically acceptable carrier.

Additional objects of the invention will be apparent from the description which follows.

### BRIEF DESCRIPTION OF THE DRAWINGS

FIG. 1A-1F. Three dimensional structure of substrate-bound hIDO showing the presence of two small molecule binding sites (Sa and Si) and solution data demonstrating the ability of the Si site in binding the substrate Trp, an effector IDE and an uncompetitive inhibitor MitoC. In (a), the heme and its two axial ligands are labeled in magenta, the Trp bound in the Sa site is labeled in green, while the indole ethanol (IDE) bound in the Si site is labeled in cyan. The bottom inset in (la) shows the blow-up view of the specific interactions between the small molecules bound in the Sa and Si sites and their surrounding environment. (1b-1c) Activity data supporting the inhibition effect of Trp binding to the Si site, as well as a cartoon illustrating the associated mechanism. (1d) Activity data demonstrating the enhanced activity induced by hIDO binding to the Si site. (1e) Uncompetitive inhibition of hIDO induced by Mitomycin C (MitoC) binding to the Si site. (1f) Chemical structures of Trp, IDE and MitoC.

### DETAILED DESCRIPTION OF THE INVENTION

Provided is a non-transitory computer-readable medium coupled to the one or more data processing apparatus having instructions stored thereon which, when executed by the one or more data processing apparatus, cause the one or more data processing apparatus to perform a method comprising:
providing a crystal coordinate structure of the small molecule binding site on a human indoleamine 2,3-dioxygenase (hIDO) enzyme, wherein the small molecule binding site is not a hIDO active site;
contacting the small molecule binding site in silico with a candidate small organic molecule structure; and
determining if the candidate small organic molecule structure binds the small molecule binding site at a free energy level below a predetermined free energy level,
wherein a candidate small organic molecule structure that binds the small molecule binding site at a free energy level below the predetermined free energy level is identified and detected as a molecule which binds to a small molecule binding site on a human indoleamine 2,3-dioxygenase (hIDO) enzyme.

In an embodiment, the crystal coordinate structure is provided in Megatable 1, 2 or 3 as listed herein.

Also provided is a system for identifying a cognate ligand molecule for a known biological receptor comprised of amino acid residues, comprising:
one or more data processing apparatus;
a graphical user interface; and
a non-transitory computer-readable medium coupled to the one or more data processing apparatus having instructions stored thereon which, when executed by the one or more data processing apparatus, cause the one or more data processing apparatus to perform a method comprising:
providing a crystal coordinate structure of the small molecule binding site on a human indoleamine 2,3-dioxygenase (hIDO) enzyme, wherein the small molecule binding site is not a hIDO active site;
contacting the small molecule binding site in silico with a candidate small organic molecule structure; and
determining if the candidate small organic molecule structure binds the small molecule binding site at a free energy level below a predetermined free energy level,
wherein a candidate small organic molecule structure that binds the small molecule binding site at a free energy level below the predetermined free energy level is identified and detected as a molecule which binds to a small molecule binding site on a human indoleamine 2,3-dioxygenase (hIDO) enzyme.

In an embodiment, the crystal coordinate structure is provided in Megatable 1, 2 or 3 as listed herein.

Also provided is a method of detecting a molecule which binds to a small molecule binding site on a human indoleamine 2,3-dioxygenase (hIDO) enzyme, wherein the small molecule binding site is not a hIDO active site, comprising:
providing a crystal coordinate structure of the small molecule binding site on a human indoleamine 2,3-dioxygenase (hIDO) enzyme, wherein the small molecule binding site is not a hIDO active site;
contacting the small molecule binding site in silico with a candidate small organic molecule structure; and
determining if the candidate small organic molecule structure binds the small molecule binding site at a free energy level below a predetermined free energy level,
wherein a candidate small organic molecule structure that binds the small molecule binding site at a free energy level below the predetermined free energy level is identified and detected as a molecule which binds to a small molecule binding site on a human indoleamine 2,3-dioxygenase (hIDO) enzyme.

In an embodiment, the crystal coordinate structure is provided in Megatable 1, 2 or 3 as listed herein.

In an embodiment of the inventions, the candidate small organic molecule structure is an analog of tryptophan. In an embodiment of the inventions, the analog of tryptophan comprises an indole of tryptophan.

In an embodiment of the inventions, the candidate small organic molecule structure is an analog of indole ethanol.

In an embodiment of the inventions, the candidate small organic molecule structure is an analog of mitomycin C.

In an embodiment of the methods, the method further comprises contacting an isolated human indoleamine 2,3-dioxygenase (hIDO) enzyme with an amount of the candidate agent identified as binding to the small molecule binding site on the hIDO in the presence an amount of L-tryptophan and determining whether the candidate agent increases or decreases a rate of degradation of the L-tryptophan to N-formylkynurenine relative to a predetermined control rate of degradation, and identifying the candidate agent as an agonist of hIDO where the candidate agent increases the rate of degradation, or identifying the candidate agent as an antagonist of hIDO where the candidate agent decreases the rate of degradation.

Embodiments of the invention and all of the functional operations described in this specification can be implemented in digital electronic circuitry, or in computer software, firmware, or hardware, including the structures disclosed in this specification and their structural equivalents, or in combinations of one or more of them. Embodiments of the invention can be implemented as one or more computer program products, i.e., one or more modules of computer program instructions encoded on a non-transitory computer readable medium for execution by, or to control the operation of, data processing apparatus. The non-transitory computer readable medium can be a machine readable storage device, a machine readable storage substrate, a memory device, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database including a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio player, a Global Positioning System (GPS) receiver, to name just a few. Non-transitory computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the invention can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the invention can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

A non-transitory computer readable medium comprising instructions stored thereon for performing the methods described herein is also provided.

A method of activating or increasing activation of human indoleamine 2,3-dioxygenase (hIDO) enzyme comprising contacting the hIDO with an amount of Apigenin, Baicalein or Chrysin having the structures as set forth in Table 1, or a pharmaceutical salt of one thereof, in an amount effective to activating or increasing activation of hIDO.

In an embodiment, the hIDO is contacted with the amount of a pharmaceutical salt of Apigenin, Baicalein or Chrysin.

A method of effecting immunosuppression in a subject comprising administering an amount of Apigenin, Baicalein or Chrysin having the structures as set forth in Table 1, or a pharmaceutical salt of one thereof, in an amount effective to activate or increasing activation of hIDO.

In an embodiment, the hIDO is contacted with the amount of a pharmaceutical salt of Apigenin, Baicalein or Chrysin.

A method of treating an autoimmune disease or inhibiting an organ transplant rejection in a subject comprising administering an amount of Apigenin, Baicalein or Chrysin having the structures as set forth in Table 1, or a pharmaceutical salt of one thereof, in an amount effective to treat an autoimmune disease or inhibit an organ transplant rejection.

In an embodiment, the hIDO is contacted with the amount of a pharmaceutical salt of Apigenin, Baicalein or Chrysin. In an embodiment, the autoimmune disease is treated. In an embodiment, the organ transplant rejection is inhibited. In an embodiment, the Apigenin, Baicalein or Chrysin is administered parenterally.

Autoimmune diseases treatable by the invention include acute disseminated encephalomyelitis (ADEM), alopecia areata, antiphospholipid syndrome, autoimmune cardiomyopathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticarial, autoimmune uveitis, Behçet's disease, celiac disease, Chagas disease, cold agglutinin disease, Crohn's disease, dermatomyositis, diabetes mellitus type 1, eosinophilic fasciitis, gastrointestinal pemphigoid, Goodpasture's syndrome, Grave's syndrome, Guillain-Barré syndrome, Hashimoto's encephalopathy, Hashimoto's thyroiditis, lupus erythematosus, Miller-Fisher syndrome, mixed connective tissue disease, myasthenia gravis, pemphigus vulgaris, pernicious anaemia, polymyositis, psoriasis, psoriatic arthritis, relapsing polychondritis, rheumatoid arthritis, rheumatic fever, Sjogren's syndrome, temporal arteritis, transverse myelitis, ulcerative colitis, undifferentiated connective tissue disease, vasculitis, Wegener's granulomatosis.

A pharmaceutical composition for activating or increasing activation of human indoleamine 2,3-dioxygenase comprising an amount of Apigenin, Baicalein or Chrysin having the structures as set forth in Table 1, or a pharmaceutical salt of one thereof, and a pharmaceutically acceptable carrier.

Administration can be auricular, buccal, conjunctival, cutaneous, subcutaneous, endocervical, endosinusial, endotracheal, enteral, epidural, via hemodialysis, interstitial, intrabdominal, intraamniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronary, intradermal, intradiscal, intraductal, intraepidermal, intraesophagus, intragastric, intravaginal, intragingival, intraileal, intraluminal, intralesional, intralymphatic, intramedullary, intrameningeal, intramuscular, intraocular, intraovarian, intraepicardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intraventricular, intravesical, intravitreal, laryngeal, nasal, nasogastric, ophthalmic, oral, oropharyngeal, parenteral, percutaneous, periarticular, peridural, rectal, inhalationally, retrobulbar, subarachnoid, subconjuctival, sublingual, submucosal, topically, transdermal, transmucosal, transplacental, transtracheal, ureteral, uretheral, and vaginal.

The compounds of the instant invention may be in a salt form. As used herein, a “salt” is salt of the instant compounds which has been modified by making acid or base, salts of the compounds. In the case of compounds used for treatment of a disease, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci . 66:1-19).

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### Experimental Details

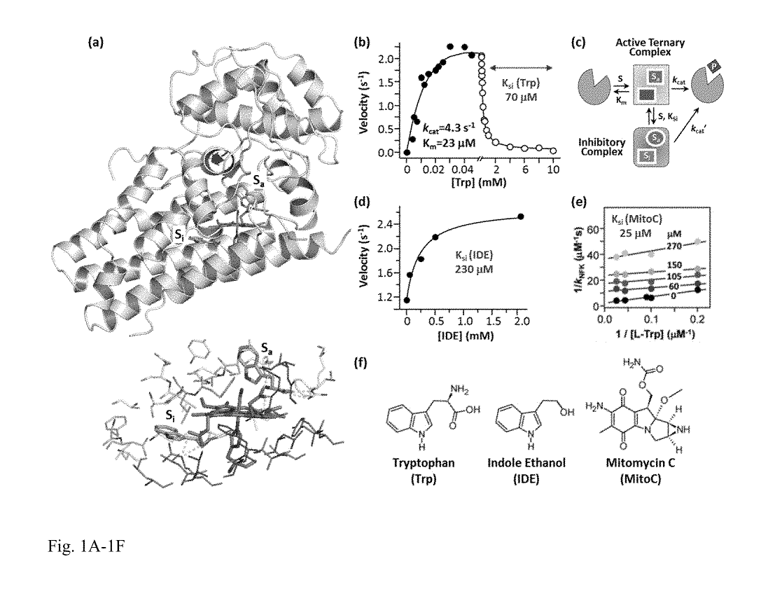

The crystal structure of hIDO was first solved in 2006, based on two substrate-free complexes (5). Subsequently, several additional inhibitor-bound structures were reported (6,7). However, until now drug design has targeted the active site. Herein the inventors have solved the crystal structures of substrate-bound complexes of hIDO for the first time. In addition to the active site (Sa), which binds the substrate tryptophan (Trp), a previously unknown small molecule binding site (Si) is identified herein which is, in the physiological context, occupied by either a second substrate, Trp, or an effector, indole ethanol (IDE) (FIG. 1a).

The existence of the Si site in hIDO was first proposed by us in 2009 based on solution studies (8). Our solution data indicate that the binding of Trp to the Si site inhibits the enzyme activity (FIG. 2b-c) (8), while the binding of the effector IDE to the Si site promotes it (FIG. 2d) 8. In addition, the data suggest that the Si site can bind an uncompetitive inhibitor MitoC, thereby inhibiting the enzyme activity (with a Ki of 25 μM, FIG. 2e) 8. It is notable that for decades MitoC has been widely used as a chemotherapy drug for several types of cancer (9). Our data demonstrate that hIDO is a target of MitoC that at least partially accounts for its therapeutic effect.

The possibility that an Si site in hIDO might exist was controversial due to the lack of direct structural evidence supporting it. New crystallographic data obtained herein shows that an Si site in hIDO does indeed exist, and also provides the three-dimensional structural coordinate data of the Si site that can be used to identify structural analogs of IDE, Trp and MitoC, as agonists or antagonists for hIDO, which can be used alone or in combination with Sa site inhibitors for treating cancer or suppressing organ transplantation rejection. Obtaining this data was a demanding task. Obtaining crystals of the enzyme, in which the target site is occupied, is a challenging task. Two mutants of the enzyme were designed and, in concert with use of an effector (IDE), establishing the existence and identification of the site was finally achieved. The wild type enzyme was used as well as two mutants: F270G and S167H. Megatables 1, 2 and 3, incorporated by reference herein, contain the full crystal structural data and are .txt versions of Protein Databank (PDB) files of (i) the wild type enzyme, (ii) a mutant (S167H), and (iii) a second mutant, (F270G). It was demonstrated that in the wild type enzyme, IDE bound in the novel target site, Si; in the F270G mutant, Trp bound in the Si site, and in the F270G mutant, IDE could bind the Si site also.

TABLE 1
Examples of compounds tested for activity at the Si site by Xtal/docking.

| Activity type | Molecule name | Structure |
|---|---|---|
| Inhibitor | Pfizer (PF068400003) | 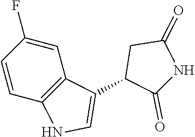 [SMILES: O=C1C[C@@H](c2c[nH]c3ccc(F)cc23)C(=O)N1] |
| Inhibitor | Ark 1 (2-Benzofuran- 3-yl-ethylamine) | 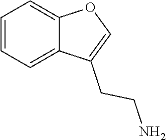 [SMILES: NCCc1coc2ccccc12] |
| Effector | IDE | 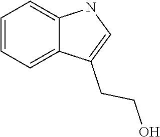 [SMILES: OCCc1c[nH]c2ccccc12] |
| Effector | Apigenin | 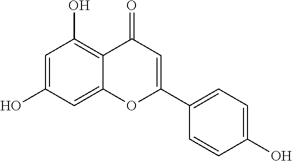 [SMILES: O=c1cc(-c2ccc(O)cc2)oc2cc(O)cc(O)c12] |
| Effector | Baicalein | 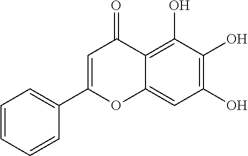 [SMILES: O=c1cc(-c2ccccc2)oc2cc(O)c(O)c(O)c12] |
| Effector | Chrysin | 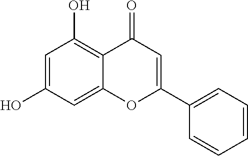 [SMILES: O=c1cc(-c2ccccc2)oc2cc(O)cc(O)c12] |

A number of compounds were tested for Si activity using the methods presented herein. Pfizer (PF068400003) and Ark1 (2-Benzofuran-3-yl-ethylamine), not previously known to act on the Si site (as opposed to the Sa site) indicated inhibitory activity. IDE showed effector activity. Apigenin, Baicalein, and Chrysin, none previously known to have hIDO effects, were all determined to be effectors at the hIDO Si site.

### REFERENCES

1. Uyttenhove, C. et al. Evidence for a tumoral immune resistance mechanism based on tryptophan degradation by indoleamine 2,3-dioxygenase. Nat Med 9, 1269-74 (2003).
2. Munn, D. H. & Mellor, A. L. Indoleamine 2,3 dioxygenase and metabolic control of immune responses. Trends Immunol 34, 137-43 (2013).
3. Sheridan, C. IDO inhibitors move center stage in immuno-oncology. Nat Biotechnol 33, 321-2 (2015).
4. Rohrig, U. F., Majjigapu, S. R., Vogel, P., Zoete, V. & Michielin, O. Challenges in the Discovery of Indoleamine 2,3-Dioxygenase 1 (IDO1) Inhibitors. J Med Chem 58, 9421-37 (2015).
5. Sugimoto, H. et al. Crystal structure of human indoleamine 2,3-dioxygenase: catalytic mechanism of 02 incorporation by a heme-containing dioxygenase. Proc Natl Acad Sci U S A 103, 2611-6 (2006).
6. Tojo, S. et al. Crystal Structures and Structure-Activity Relationships of Imidazothiazole Derivatives as IDO1 Inhibitors. ACS Med Chem Lett 5, 1119-23 (2014).
7. Peng, Y. H. et al. Important Hydrogen Bond Networks in Indoleamine 2,3-Dioxygenase 1 (IDO1) Inhibitor Design Revealed by Crystal Structures of Imidazoleisoindole Derivatives with IDO1. J Med Chem 59, 282-93 (2016).
8. Lu, C., Lin, Y. & Yeh, S. R. Inhibitory substrate binding site of human indoleamine 2,3-dioxygenase. J Am Chem Soc 131, 12866-7 (2009).
9. Carter, S. K. & Crooke, S. T. (eds.). Mitomycin C: current status and new developments, (Academic Press, New York, 1979).

1. A non-transitory computer-readable medium coupled to the one or more data processing apparatus having instructions stored thereon which, when executed by the one or more data processing apparatus, cause the one or more data processing apparatus to perform a method comprising:
providing a crystal coordinate structure of the small molecule binding site on a human indoleamine 2,3-dioxygenase (hIDO) enzyme, wherein the small molecule binding site is not a hIDO active site;
contacting the small molecule binding site in silico with a candidate small organic molecule structure; and
determining if the candidate small organic molecule structure binds the small molecule binding site at a free energy level below a predetermined free energy level,
wherein a candidate small organic molecule structure that binds the small molecule binding site at a free energy level below the predetermined free energy level is identified and detected as a molecule which binds to a small molecule binding site on a human indoleamine 2,3-dioxygenase (hIDO) enzyme.

2. A system for identifying a cognate ligand molecule for a known biological receptor comprised of amino acid residues, comprising:
one or more data processing apparatus;
a graphical user interface; and
a non-transitory computer-readable medium coupled to the one or more data processing apparatus having instructions stored thereon which, when executed by the one or more data processing apparatus, cause the one or more data processing apparatus to perform a method comprising:
providing a crystal coordinate structure of the small molecule binding site on a human indoleamine 2,3-dioxygenase (hIDO) enzyme, wherein the small molecule binding site is not a hIDO active site;
contacting the small molecule binding site in silico with a candidate small organic molecule structure; and
determining if the candidate small organic molecule structure binds the small molecule binding site at a free energy level below a predetermined free energy level,
wherein a candidate small organic molecule structure that binds the small molecule binding site at a free energy level below the predetermined free energy level is identified and detected as a molecule which binds to a small molecule binding site on a human indoleamine 2,3-dioxygenase (hIDO) enzyme.

3. A method of detecting a molecule which binds to a small molecule binding site on a human indoleamine 2,3-dioxygenase (hIDO) enzyme, wherein the small molecule binding site is not a hIDO active site, comprising:
providing a crystal coordinate structure of the small molecule binding site on a human indoleamine 2,3-dioxygenase (hIDO) enzyme, wherein the small molecule binding site is not a hIDO active site;
contacting the small molecule binding site in silico with a candidate small organic molecule structure; and
determining if the candidate small organic molecule structure binds the small molecule binding site at a free energy level below a predetermined free energy level,
wherein a candidate small organic molecule structure that binds the small molecule binding site at a free energy level below the predetermined free energy level is identified and detected as a molecule which binds to a small molecule binding site on a human indoleamine 2,3-dioxygenase (hIDO) enzyme.

4. The method of claim 3, wherein the candidate small organic molecule structure is an analog of tryptophan.

5. The method of claim 4, wherein the analog of tryptophan comprises an indole of tryptophan.

6. The method of claim 3, wherein the candidate small organic molecule structure is an analog of indole ethanol.

7. The method of claim 3, wherein the candidate small organic molecule structure is an analog of mitomycin C.

8. The method of claim 3, further comprising contacting an isolated human indoleamine 2,3-dioxygenase (hIDO) enzyme with an amount of the candidate agent identified as binding to the small molecule binding site on the hIDO in the presence an amount of L-tryptophan and determining whether the candidate agent increases or decreases a rate of degradation of the L-tryptophan to N-formylkynurenine relative to a predetermined control rate of degradation, and identifying the candidate agent as an agonist of hIDO where the candidate agent increases the rate of degradation, or identifying the candidate agent as an antagonist of hIDO where the candidate agent decreases the rate of degradation.

9. A method of activating or increasing activation of human indoleamine 2,3-dioxygenase (hIDO) enzyme comprising contacting the hIDO with an amount of Apigenin, Baicalein or Chrysin having the structures as set forth in Table 1, or a pharmaceutical salt of one thereof, in an amount effective to activating or increasing activation of hIDO.

10. The method of claim 9, wherein the hIDO is contacted with the amount of a pharmaceutical salt of Apigenin, Baicalein or Chrysin.

11. A method of effecting immunosuppression in a subject comprising administering an amount of Apigenin, Baicalein or Chrysin having the structures as set forth in Table 1, or a pharmaceutical salt of one thereof, in an amount effective to activate or increasing activation of hIDO.

12. The method of claim 11, wherein the hIDO is contacted with the amount of a pharmaceutical salt of Apigenin, Baicalein or Chrysin.

13. A method of treating an autoimmune disease or inhibiting an organ transplant rejection in a subject comprising administering an amount of Apigenin, Baicalein or Chrysin having the structures as set forth in Table 1, or a pharmaceutical salt of one thereof, in an amount effective to treat an autoimmune disease or inhibit an organ transplant rejection.

14. The method of claim 13, wherein the hIDO is contacted with the amount of a pharmaceutical salt of Apigenin, Baicalein or Chrysin.

15. The method of claim 13, wherein the autoimmune disease is treated.

16. The method of claim 13, wherein the organ transplant rejection is inhibited.

17. The method of claim 13, wherein the Apigenin, Baicalein or Chrysin is administered parenterally.

18. A pharmaceutical composition for activating or increasing activation of human indoleamine 2,3-dioxygenase comprising an amount of Apigenin, Baicalein or Chrysin having the structures as set forth in Table 1, or a pharmaceutical salt of one thereof, and a pharmaceutically acceptable carrier.
